(19) United States
(12) Patent Application Publication
(10) Pub. No.: US 2024/0000369 A1
(43) Pub. Date: Jan. 4, 2024

(54) AUTOMATIC PARKINSONS DISEASE DETECTION BASED ON THE COMBINATION OF LONG-TERM ACOUSTIC FEATURES AND MEL FREQUENCY COEFFICIENTS (MFCCs)

(71) Applicant: Imam Abdulrahman Bin Faisal University, Dammam (SA)

(72) Inventors: Raneem Ahmed ALQAHTANI, Dammam (SA); Sara Ali AL-QAHTANI, Dammam (SA); Dannah Safran ALSAFRAN, Dammam (SA); Lola El SAHMARANY, Dammam (SA); Maram ALQARNI, Dammam (SA)

(73) Assignee: Imam Abdulrahman Bin Faisal University, Dammam (SA)

(21) Appl. No.: 17/853,044

(22) Filed: Jun. 29, 2022

(51) Int. Cl. A61B 5/00; G06N 5/00

(52) U.S. Cl. CPC A61B 5/4082; A61B 5/4803; A61B 5/7267; A61B 5/7203; A61B 5/7225; A61B 5/7257; A61B 5/7221; G06N 5/003

(57) ABSTRACT

A system, method, and non-transitory computer readable medium for discriminating between patients with neurodegenerative disease and healthy patients. The method includes obtaining a first plurality of voice signals from known healthy humans and known neurogenerative diseases humans, extracting long-term acoustic features of the first plurality of voice signals, extracting Mel frequency coefficients (MFCCs) from the first plurality of voice signals, creating a set A of short-term acoustic features based on the MFCCs, performing a backward stepwise selection to create a set B of long-term acoustic features and a set C, where set C includes the features of set B combined with the features of set A, creating a random forest classification model, obtaining a second plurality of voice signals from humans of undetermined health status, and applying the second plurality of voice signals against the random forest classification model to determine which patients are neurodegenerative diseased patients.

### BACKGROUND

### Technical Field

The present disclosure is directed to detection of Parkinson's and other neurodegenerative diseases based on long-term acoustic features and Mel frequency coefficients (MFCCs).

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Parkinson's disease is one of the common neurodegenerative diseases. People suffering from Parkinson's disease experience two types of symptoms namely motor symptoms and non-motor symptoms, caused by chronic degeneration of dopaminergic neurons in the brain. Multiple screening tests are conducted to detect Parkinson's disease. Traditionally, these screening tests focus mainly on motor symptoms, such as tremors, muscle rigidity, and gait disturbances.

However, motor symptoms are detectable only after degeneration of 70% of the neurons. Further, it is evident from several studies that some non-motor symptoms such as dysphagia, incontinence, and vocal impairment appear long before the motor symptoms. Early detection of Parkinson's disease is a key to preventing excessive degeneration of neurons and slowing the progression of Parkinson's disease. Therefore, it is preferred to detect Parkinson's disease at an early stage by screening for non-motor symptoms, allowing proactive and preventative medical treatment of a person diagnosed with Parkinson's disease.

Vocal impairment is one of the earliest symptoms experienced by 90% of the patients with Parkinson's disease thus leading use of vocal biomarkers to diagnose Parkinson's. A vocal biomarker extracts acoustic features from speech of a person who is to be tested and compares the extracted acoustic features to a library of such features for detecting Parkinson's disease or predicting the severity of Parkinson's disease. However, requiring a high correlation between the extracted acoustic features, results in inaccurate prediction due to recording of voice mostly in noisy environments.

Accordingly, it is one object of the present disclosure to provide a system and a method for detection of Parkinson's disease in an accurate and efficient manner.

### SUMMARY

In an exemplary embodiment, a machine-learning method to differentiate between patients with neurodegenerative disease and healthy patients is disclosed. The method includes obtaining a first plurality of voice signals from known healthy humans and known neurogenerative diseases humans, extracting one or more long-term acoustic features of the first plurality of voice signals, extracting Mel frequency coefficients (MFCCs) from the first plurality of voice signals, creating a set A of short-term acoustic features based on the MFCCs, performing a backward stepwise selection of the long-term acoustic features to create a set B of long-term acoustic features and a set C, set C comprising the set B of long-term acoustic features combined with the set A of short-term acoustic features, creating a random forest classification model by using sets A, B, and C in order to classify healthy patients and neurodegenerative diseases patients, obtaining a second plurality of voice signals from humans of undetermined health status, and applying the second plurality of voice signals against the random forest classification model in order to determine which patients in the second plurality of voice signals are healthy patients and which are neurodegenerative diseased patients.

In another exemplary embodiment, a medical diagnostic system includes one or more processors, a memory, a microphone, and a circuitry. The circuitry is configured to: obtain a first plurality of voice signals from known healthy humans and known neurogenerative diseases humans, extract one or more long-term acoustic features of the first plurality of voice signals, extract Mel frequency coefficients (MFCCs) from the first plurality of voice signals, create a set A of short-term acoustic features based on the MFCCs, perform a backward stepwise selection of the long-term acoustic features to create a set B of long-term acoustic features and a set C, set C comprising the set B of long-term acoustic features combined with the set A of short-term acoustic features, configure a random forest classification model by using set C in order to classify healthy patients and neurodegenerative diseases patients, obtain a second plurality of voice signals from humans of undetermined health status, and apply the second plurality of voice signals against the model in order to determine which patients in the second plurality of voice signals samples are healthy patients and which are neurodegenerative diseases patients.

In another exemplary embodiment, a non-transitory computer-readable storage medium storing computer-readable instructions that, when executed by one or more processors, cause the one or more processors to obtain a first plurality of voice signals from human patients, extract one or more long-term acoustic features of the voice signals, extract Mel frequency coefficients (MFCCs) from the voice signals, creating a set A of short-term acoustic features based on the MFCCs, perform a backward stepwise selection of long-term acoustic features to create a set B of long term acoustic features and a set C, set C comprising long-term acoustic features combined with the set A of short-term acoustic features, create a random forest classification model by using sets A, B, and C in order to create a classification of healthy patients and neurodegenerative diseases patients, obtain a second plurality of voice signals, and apply the second plurality of voice signals against the model in order to determine which of the second plurality of voice signals are from healthy patients and which are from neurodegenerative diseases patients.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a block diagram of a medical diagnostic system, according to aspects of the present disclosure;

FIG. 2 depicts a flow diagram of an example of Parkinson's disease (PD) detection via vocal feature extraction, according to aspects of the present disclosure;

FIG. 3 illustrates a method of extracting coefficients of Mel spectrum, according to aspects of the present disclosure;

FIG. 4 represents a schematic working of a random forest classification model, according to aspects of the present disclosure;

FIG. 5 illustrates a method of discriminating between patients with neurodegenerative disease and healthy patients, according to aspects of the present disclosure;

FIG. 6 represents receiver operating characteristics (ROC) for set A, set B, and set C, according to aspects of the present disclosure;

FIG. 7 is an illustration of a non-limiting example of details of computing hardware used in the computing system, according to aspects of the present disclosure;

FIG. 8 is an exemplary schematic diagram of a data processing system used within the computing system, according to aspects of the present disclosure;

FIG. 9 is an exemplary schematic diagram of a processor used with the computing system, according to aspects of the present disclosure; and

FIG. 10 illustrates a non-limiting example of distributed components that may share processing with the controller, according to aspects of the present disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a medical diagnostic system and a machine-learning method to differentiate between patients with neurodegenerative disease and healthy patients. The disclosed method and system employ a random forest classification model to improve Parkinson's disease detection. The random forest classification model is configured to use a combination of long-term features and Mel frequency cepstral coefficients (MFCCs). The disclosed method and system use three sets of input: MFCCs features (set A), long-term features (set B), and a combination of MFCCs features with long-term features (set C). The comparison among results of the three sets (set A, set B, and set C) indicates that the set C (combined features) has improved detection accuracy to 88.84% while the accuracy for MFCCs features, and long-term features non-combined sets are 84.12% and 84.00% respectively. Set C was less correlated and more robust in the presence of noise than sets A and B. Hence, set C achieved the highest accuracy of 88.84%. Thereby, the present disclosure improves the accuracy of Parkinson's disease detection and allows for proactive medical interventions to prevent the progression of disease. Further, the present method and system improve the reliability and effectiveness in detecting Parkinson's disease at early stages and subsequently assist in preventing its progression.

In various aspects of the disclosure, non-limiting definitions of one or more terms that will be used in the document are provided below.

A term “Mel frequency cepstral coefficients (MFCCs)” may be defined as coefficients that collectively make up a Mel frequency cepstral (MFC) that used in speech recognition and automatic speech. MFCCs is the widely used technique for extracting the features from the audio signal. In this disclosure, the term MFCC is used interchangeably with “short term features” or “short-term acoustic features.”

As used herein, the term “microphone” (colloquially a “mic” or “mike”) is an acoustic-to-electric transducer or sensor that converts sound/voice (e.g., acoustic energy) into an electrical signal (e.g., electrical energy). The microphone may include accessories such as a “lollipop” shaped filter mounted on or near the microphone to remove background noise or may include a headset and may additionally include a windshield, a foam cover, or a “Pop filter”. In one configuration, a “Pop filter”, a mesh filter to limit popping noise, is positioned between the microphone and the speaker. In addition, associated software or hardware may include background noise suppression or background noise reduction. In some embodiments, the “microphone” may actually be a two microphone system with one microphone directed to convert a human voice and a second microphone directed to recording ambient noise. The system may then remove the ambient noise from the human voice signal. Processing of the human voice signal may additional include band-pass or band-reject filtering to remove background noise.

In a preferred embodiment of the invention the microphone is a component of a multi-microphone headset system. A first microphone is mounted on an extension of the headset such that the first microphone is suspended in front of a subject at a distance of 0.5-2 inches from the lips of the subject. The extension on which the first microphone is mounted is directly connected to the headset which may optionally include earphone speakers or ear buds. The headset includes at least one second microphone configured to lay flat on a skin surface of the subject. The second microphone is preferably positioned on at least one temple of the subject. In this position, in direct contact with the skin of the subject, the second microphone obtains and permits recording of a second voice signal in the form of vibrations transmitted through the subject's oral cavity. Preferably the headset includes a matching set of skin-mounted microphones on both the right and left temples of the subject. The second microphones are connected to the first microphone through an adjustable mechanical headset device.

The second microphones function to obtain a second voice. The second voice signal may be separately processed and compared with the first voice signal obtained from the first microphone mounted in front of the subject's lips. Feature comparison of the first and second voice signals may be accomplished by mapping one or more of a set A, a set B or set C of features obtained from the first and second microphones signal (see further discussion herein).

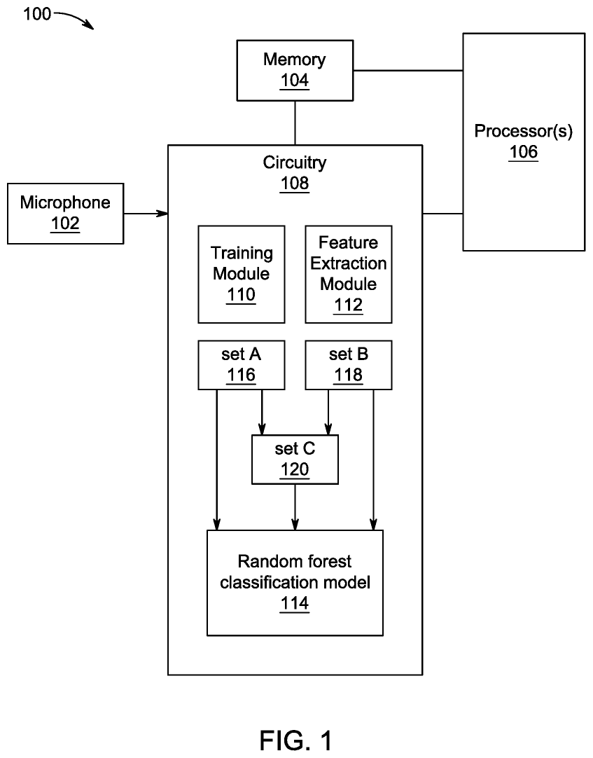

FIG. 1 illustrates a block diagram of a medical diagnostic system 100 for discriminating/differentiating patients with neurodegenerative disease and healthy patients (patients who do not have a neurodegenerative disease), according to one or more aspects of the present disclosure.

Referring to FIG. 1, the medical diagnostic system 100 (hereinafter referred to as “system 100”) includes various components such as a microphone 102, a memory 104, one or more processors 106, and a circuitry 108. In an aspect, the components of the system 100 may be suitably combined in a single chip or disposed on a same circuit board. In some other embodiments, the components are implemented on separate chips. In some embodiment, the various components of the system 100 may reside on a single computer, or they may be distributed across several computers in various arrangements. The microphone 102 is configured to receive an audio input from a person and to generate a voice signal. In an aspect, the microphone 102 may be configured to record the received audio input. The microphone 102 can be remotely placed from the system 100 and can transmit the generated voice signal to the circuitry 108 over a network. In an aspect, the microphone 102 includes communication capabilities (e.g., through cellular, Bluetooth, hotspot and/or Wi-Fi) allowing communication with the circuitry 108 and/or a centralized server. In another aspect, the network can be a public network, such as the Internet, or a private network such as an LAN or WAN network, or any combination thereof. The network can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The circuitry 108 is configured to receive or collect the transmitted voice signal (s) from the microphone 102 over the network. The circuitry 108 is coupled to the memory 104, and the one or more processor 106. In an aspect, the received voice signal is filtered by a filter, coupled with the circuitry 108, that removes frequency components that are of non-interest. This might include, for example, impulse noise such as pops and clicks, broadband noise such as buzzing and hissing, or narrow band noise as may be caused by improper grounding of the recording equipment. Other irregular noise may include traffic noise, rain, or thunder in the background. The filtered voice signal is then sampled and digitized by an analog to digital converter and the digitized samples are stored in the memory 104.

The circuitry 108 may be any device, such as Integrated Chip (IC), a desktop computer, a laptop, a tablet computer, a smartphone, a smart watch, a mobile device, a Personal Digital Assistant (PDA) or any other computing device including customized device therefor. According to an aspect, the circuitry 108 may facilitate discrimination between patients with neurodegenerative disease and healthy patient/person.

Further, the memory 104 is configured to store program instructions. In an aspect, the memory 104 is configured to store the voice signals received from the microphone 102 and the circuitry 108. In an aspect, the memory 104 is configured to store a ML model and a training set for training the ML model. The stored program instructions include a program that implements a supervised machine-learning classification model using a Random Forest classification method. Random forest is one of the most robust classifiers used for PD detection. Compared to other supervised learning classifiers, Radom Forest exhibits more resistance to over- and underfitting and less sensitivity to outliers, with relatively fewer hyper-parameters which are produced by n train subsets. Random forest requires splitting the dataset into train and test sets, where the train set is used to build the model and the latter is used to test the model's performance. The combination of parameters producing the smallest error is chosen for classification. The Random Forest Classification method is used to differentiate between patients with neurodegenerative disease and healthy patients and may implement other embodiments described in this specification. The training set includes a first plurality of voice signals of known healthy humans and known neurogenerative diseased humans. The training set further contains extracted voice features including long term features (e.g., intensity parameters, formant frequencies, bandwidth parameters, and vocal fold parameters), short-term features (MFCCs), and similar other scope features. In an aspect, the training set is configured to auto update by adding the received voice signals. The memory 104 may include any computer-readable medium known in the art including, for example, volatile memory, such as Static Random Access Memory (SRAM) and Dynamic Random Access Memory (DRAM) and/or nonvolatile memory, such as Read Only Memory (ROM), erasable programmable ROM, flash memories, hard disks, optical disks, and magnetic tapes.

The processor(s) 106 may be configured to fetch and execute computer-readable instructions stored in the memory 104. According to an aspect of the present disclosure, the processor 106 may be implemented as one or more microprocessors, microcomputers, microcontrollers, digital signal processors, central processing units, state machines, logic circuitries, and/or any devices that manipulate signals based on operational instructions.

In an exemplary aspect, the circuitry 108 is configured to obtain the first plurality of voice signals from known healthy humans and known neurogenerative diseases humans fetched from the memory 104. In some aspect, the circuitry 108 includes a training module 110, a feature extraction module 112, and a random forest classification model (RF model) 114.

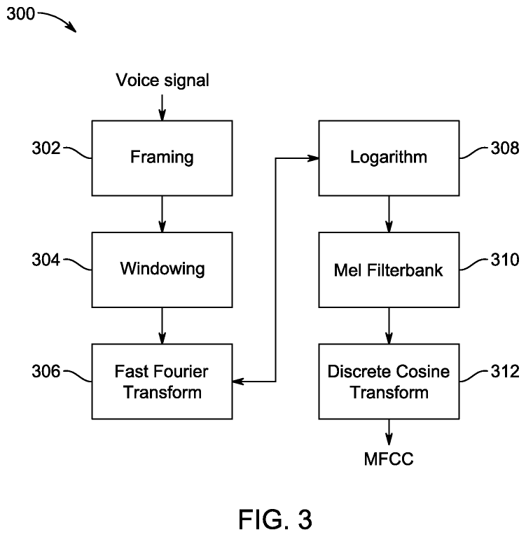

In principle, the ML model is a model which created by the ML and may be trained in a training phase based on a set of labelled training data. After the training phase, the ML model is configured to apply the learning to the received voice signals. The training module 110 is configured to cooperate with the memory 104 to receive information related to the stored voice signals. The training module 110 trains one or more machine learning models using the training set obtained from the memory 104. The training module 110 is configured to train the RF model 114 to differentiate between patients with neurodegenerative disease and healthy patients based on the received information/voice signals. As the name implies, the RF model applies bootstrap sampling to produce multiple decision trees (DT) which are produced by n train subsets, as illustrated in FIG. 3.

The hyper-parameter n is indicative of the number of DT constituting the RF model. Typically, a larger forest leads to a more robust performance. In each bootstrap set, some randomly chosen observations referred to as Out-Of-Bag (OOB) samples do not participate in tree training, instead, OOB are used as unseen test data to estimate the OOB error of each grown DT. The combination of parameters producing the smallest OOB error is chosen for classification. After building the model, the observations in the test set which are unknown to RF are evaluated and each decision tree in the forest produces a vote and the majority vote is selected as the forest final classification.

Using the feature extraction module 112, the circuitry 108 is configured to extract the acoustic features of the received voice signals. Initially, the circuitry 108 extracts the acoustic features of the first plurality of voice signals. During the feature extraction mainly two types of acoustic features are extracted, namely as long-term features, and short-term features. The circuitry 108 is configured to extract one or more long-term features including any of: a relative average perturbation, a jitter, an amplitude perturbation quotient, a shimmer, a detrended fluctuation analysis, a minimum intensity, a maximum intensity, a mean intensity, and a formant frequency.

Long-term features are dependent on the behavior of signal in terms of amplitude and frequency at certain points in time [described in M. Little, P. McSharry, E. Hunter, J. Spielman, and L. Ramig, “Suitability of dysphonia measurements for telemonitoring of parkinson's disease,” Nature Precedings, pp. 1-1, 2008 included herein by reference]. For the disclosed method, nine long-term features are used; relative average perturbation (RAP):Jitter, local absolute jitter, amplitude perturbation quotient (APQ3): Shimmer, detrended fluctuation analysis (DFA), minimum intensity, maximum intensity, mean intensity, and formant frequencies F1, and F2. Jitter is a measure of frequency perturbation per cycle that indicates the vibratory stability of vocal cords which may be compromised for PD patients; therefore, jitter values are relatively higher for PWP. RAP: Jitter measures the difference in absolute average frequency perturbation between any two consecutive cycles while local absolute jitter refers to the average absolute difference between one period and its two neighboring periods. Shimmer(apq3) is a long-term feature that measures the amplitude perturbation per cycle throughout three consecutive periods [described by J. P. Teixeira, C. Oliveira, and C. Lopes, “Vocal acoustic analysis— jitter, shimmer and HNR parameters,” Procedia Technology, vol. 9, pp. 1112-1122, 2013 included herein by reference]. Parkinsonian voices are described as monopitch where amplitude variations are almost nonexistent, consequently, shimmer values for PWP are relatively low. DFA measures the non-stationary long-term auto-correlation of the signal using a scaling exponent that expresses the magnitude of correlation. Pathological voices of people with Parkinson's disease possess relatively higher values for the exponent as a result of vocal impairment [described by C. Bhattacharyya, S. Sengupta, S. Nag, S. Sanyal, A. Banerjee, R. Sengupta, and D. Ghosh, “Acoustical classification of different speech acts using nonlinear methods,” arXiv preprint arXiv:2004.08248, 2020 included herein by reference]. Parkinson's disease patients suffer from a condition called hypophonia characterized by volume weakness, so measures of intensity are important to increase the discriminative potential between healthy subjects and PWP. The proposed method utilizes minimum, maximum, and mean intensities to quantify the strength of vocal fold vibration and magnitude of volume production. Minimum and maximum intensities describe the intensity variations, while mean intensity correlates with the perception of vocal loudness. A high value of intensity indicates loudness and vice versa. Vocal intensities of healthy people range from 70 to 80 dB and around dB for PD patients [described by D. Abur, A. A. Lupiani, A. E. Hickox, B. G. Shinn-Cunningham, and C. E. Stepp, “Loudness perception of pure tones in parkinson's disease,” Journal of speech, language, and hearing research, vol. 61, no. 6, pp. 1487-1496, 2018 included herein by reference].

In a working aspect, the circuitry 108 is configured to extract Mel frequency coefficients (MFCCs) from the received first plurality of voice signals and create a set A 116 of short-term acoustic features based on the extracted MFCCs. In an aspect, to extract the MFCCs the circuitry 108 is configured to employ following exemplary steps:
dividing the voice signal into overlapping frames, where each frame contains a plurality of samples and wherein the overlap is between 30% and 50% of the frame;
windowing the overlapping frames where size of the window is 20-40 ms;
applying a Fast Fourier Transform (FFT) to convert the voice signal to a frequency domain;
calculating logarithms of average values of a spectral power density in each of the frames to model the voice signal in a cepstral domain;
creating Mel filterbanks within the cepstral domain; and
performing a discrete cosine transformation (DCT) on the Mel filterbanks to calculate the MFCCs.

The backward stepwise selection (BSWS) (for example, using a feature selection algorithm) is applied to the extracted acoustic features. The BSWS is configured to reduce the dimensionality of feature subsets and subsequently reduce the computational resources required for selecting the optimal feature set. The circuitry 108 is configured to perform the BSWS on the long-term acoustic features to create a set B 118 of long-term acoustic features. The circuitry 108 may be configured to obtain and apply the BSWS to create a set C 120. The set C 120 includes the long-term acoustic features of set B 118 in combination with the short-term acoustic features of set A 116. In an aspect, the circuitry 108 is configured to calculate the BSWS of the long-term acoustic features by performing following steps:
starting with a model with a full set of long-term acoustic features;
iteratively removing a particular feature that has the least significance for model accuracy;
determining if a removal of a particular feature resulted in improving model performance wherein performance is measured by an accuracy, a specificity, a sensitivity, or an area under a curve, and removing the particular feature from the model;
determining if a removal of the particular feature resulted in worsening the model's performance and returning the particular feature to the model; and
repeating removal of each feature in the set until the best model accuracy is found.

In communication with the training module 110, the circuitry 108 is configured to create the RF model 114 by combining all features associated with set C 120 in order to classify healthy patients and neurodegenerative diseases patients. In an aspect, the RF model 114 is created by the circuitry 108 by performing following exemplary steps:
dividing the first plurality of voice signals into a training set and a test set of voice signals;
using bootstrap sampling of the training set wherein the model comprises multiple decision trees produced by multiple training subsets; and
testing the RF model 114 with the test set of voice signals.

In an operative aspect of the present system 100, to test whether a human/patient has neurodegenerative disease or whether the human/patient is healthy (considered as “healthy patient”), the circuitry 108 is configured to obtain and record a second plurality of voice signals from the human/patient, through the microphone 102. In an aspect, the second plurality of voice signals may include inputs from more than one testing human. The circuitry 108 is configured to apply the second plurality of voice signals against the RF model 114 to determine whether the testing human has neurodegenerative disease or not.

In an illustrative aspect, the neurodegenerative disease is selected from dementia, amyotrophic lateral sclerosis (ALS), Alzheimer's disease, multiple sclerosis, juvenile parkinsonism, striatonigral degeneration, progressive supranuclear palsy, pure akinesia, prion disease, corticobasal degeneration, chorea-acanthocytosis, benign hereditary chorea, paroxysmal choreoathetosis, essential tremor, essential myoclonus, Tourette Syndrome, Rett syndrome, degenerative ballism, dystonia musculorum deformans, athetosis, spasmodic torticollis, Meige syndrome, cerebral palsy, Wilson's disease, Segawa's disease, Hallervorden-Spatz syndrome, neuroaxonal dystrophy, pallidal atrophy, spinocerebellar degeneration, cerebral cortical atrophy, Holmes-type cerebellar atrophy, olivopontocerebellar atrophy, hereditary olivopontocerebellar atrophy, Joseph disease, dentatorubrop alli doluy si an atrophy, Gerstmann-Straus sl er-S cheinker syndrome, Friedreich ataxia, Roussy-Levy syndrome, May-White syndrome, congenital cerebellar ataxia, periodic hereditary ataxia, ataxia telangiectasia, amyotrophic lateral sclerosis, progressive bulbar palsy, spinal progressive muscular atrophy, spinobulbar muscular atrophy, Werdnig-Hoffmann disease, Kugelberg-Welander disease, hereditary spastic paraplegia, syringomyelia, syringobulbia, Arnold-Chiari malformation, stiff man syndrome, Klippel-Feil syndrome, Fazio-Londe disease, low myelopathy, Dandy-Walker syndrome, spina bifida, Sjogren-Larsson syndrome, radiation myelopathy, age-related macular degeneration, and cerebral apoplexy due to cerebral hemorrhage and/or dysfunction or neurologic deficits associated therewith. Other neurodegenerative diseases that are not described here are contemplated herein. The system and methods of this disclosure could also apply to multiple different vocal or non-vocal diseases, given that the appropriate features are selected for each independent disease, and said features are programmed to be extracted from the voice sample provided by the patient.

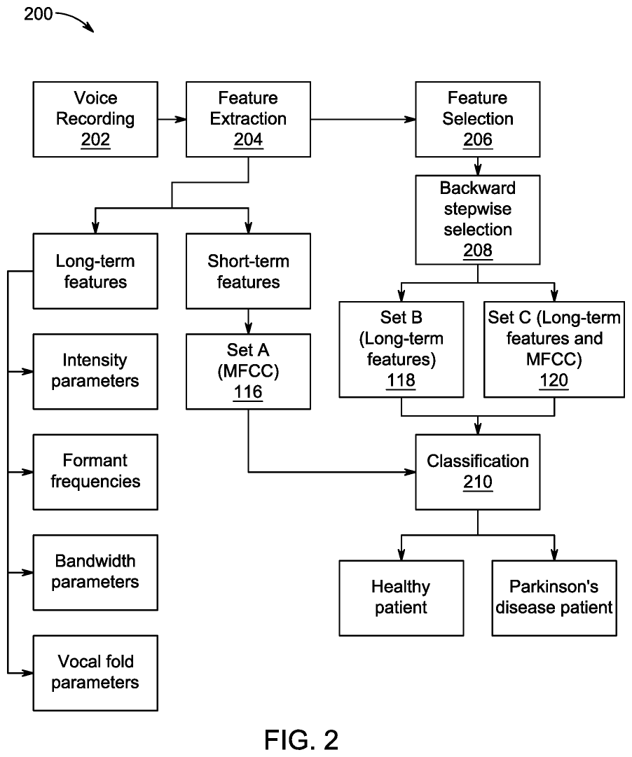

FIG. 2 illustrates a detailed flow diagram 200 of an example of Parkinson's disease (PD) detection via vocal feature extraction. As shown in FIG. 2, block 202 indicates voice recording of the patient. In an aspect, the voice of the patient is recorded by the microphone 102. A set of recorded voice of known healthy humans and known neurogenerative diseased humans is stored in the memory 104 as the first plurality of voice signals. Further, the microphone 102 is configured to record the second plurality of voice signals from humans under examination. The microphone 102 is coupled to the memory 104 to store the recorded second plurality of voice signals.

After recording the voice from the patient by the microphone 102, the acoustic features are extracted from the recorded voices using the circuitry 108. Voice production involves coordination between the motor and neurological functions of larynx. The impairment of the motor and neurological functions by laryngeal pathologies (LP) affects the production mechanism and quality of voice. Voice signals render the LP effects qualitatively, however, extracted acoustic features allow for quantitative evaluation of LP effects and transform them into an understandable format. The acoustic features associated with a single voice signal may be represented by a multidimensional feature vector that contains numerical values extracted from the voice signal. In another aspect, the acoustic features are extracted based on various parameters such as intensity parameters, formant frequencies, bandwidth parameters, and vocal fold parameters, Mel frequency cepstral coefficients, as well as other features not described herein.

As shown by block 204, during feature extraction, two types of features are extracted from the recorded voice signals. The features are long-term features and short-term features.

In many existing Parkinson's disease detection systems, use of the long-term features is known. However, extracting the value of a fundamental frequency is crucial for the successful extraction of the long-term features from the recorded signals. Thus, the long-term features are dependent on the behavior of signal in terms of amplitude and frequency at certain points in time. In an aspect of the proposed disclosure, the long-term acoustic features include, but are not limited to any of a relative average perturbation (RAP), a jitter, an amplitude perturbation quotient (APQ3), a shimmer, a detrended fluctuation analysis (DFA), a minimum intensity, a maximum intensity, a mean intensity, and formant frequencies F 1, and F2 as previously described.

The jitter is a measure of frequency perturbation per cycle that indicates the vibratory stability of vocal cords which may be compromised for PD patients; therefore, the jitter values are relatively higher for People with Parkinson's disease (PWP).

The RAP measures the difference in absolute average frequency perturbation between any two consecutive cycles, while local absolute jitter refers to the average absolute difference between one period and its two neighboring periods.

The shimmer is a feature that measures the amplitude perturbation per cycle throughout three consecutive periods. The voice of PD patients is described as monopitch where the amplitude variations are almost nonexistent, consequently, shimmer values for PWP are relatively low. The DFA measures the non-stationary long-term autocorrelation of the signal using a scaling exponent a that expresses the magnitude of correlation. Pathological voices of PWP possess relatively higher values for the exponent a because of the vocal impairment. PD patients suffer from a condition called hypophonia characterized by volume weakness, so measures of intensity are important to increase the discriminative potential between healthy patient and PWP. Vocal intensities of healthy people range from 70 to 80 dB and around 66 dB for PD patients.

The medical diagnostic system 100 utilizes minimum, maximum, and mean intensities to quantify the strength of vocal fold vibration and magnitude of volume production. The minimum and maximum intensities describe intensity variations, while mean intensity correlates with perception of vocal loudness. A high value of intensity indicates loudness and vice versa. The vocal intensities of healthy people range from 70 to 80 dB and around 65.66 dB for PD patients. Also, formant frequencies called F 1 and F2 measure the energetic density around specific frequencies in the voice spectrum. The distinct values of formant frequencies are derived from the geometrical properties of the articulators in the voice and speech production system. Restricted motion of articulators caused by PD, especially of the tongue, lead to inefficient vowel formation. Consequently, high frequency formants decrease, and low frequency formants increase when compared to healthy humans.

As shown in FIG. 2, the short-term features include Mel frequency cepstral coefficients (MFCCs), which model the natural processes of the human auditory system using Mel scale. The Mel Scale is a logarithmic transformation of a signal's frequency. The sounds of equal distance on the Mel Scale are perceived to be of equal distance to humans. The MFCCs commonly used for automatic speech recognition systems and vocal impairment detection. The system 100 is configured to create the set A 116 of short-term acoustic features based on the extracted MFCCs. A negative value for the MFCCs indicates that the frequency content of the Mel filter is concentrated in the high frequency band of the filter and vice versa. The voice of PWP is characterized by increased hoarseness and breathiness, therefore Mel coefficients associated with voice of PWP are negative and larger in magnitude than a healthy patient.

As illustrated in FIG. 2, block 206 indicates selection of features from the extracted features. An aspect of the feature selection is to improve generalization given to the training set. The feature selection supports finding a subset of features with minimum redundancy and high significance. The feature selection tests performance of the proposed ML model with all possible combinations of n-features where the number of combinations is 2′. As, testing of each 2″ feature set is computationally infeasible, costly, and exhaustive, therefore computationally efficient methods such as stepwise selection techniques are considered for the feature selection. In an example of the present disclosure, some exhaustive feature selection techniques such as forward stepwise selection, backward stepwise selection, and/or wrappers selection may be employed. In some embodiments, system 100 may utilize the backward stepwise selection (BSWS) for the feature selection.

As shown by block 208 in FIG. 2, the BSWS is applied to the extracted acoustic features. The BSWS initiates with a full ML model and iteratively removes the feature that has the least significance for the model accuracy.

The BSWS may be configured to perform following exemplary steps:
1. Let Zo be the classification model 110 with the full feature set containing n features.
2. For k=0, 1, 2, . . . , n−1, iteratively analyze the performance of all n−k models:
   a. If the removal of a feature from n−k model resulted in improving the model's performance, it is permanently removed.
   b. If the removal of a feature from n−k model feature resulted in worsening the model's performance, it is returned to the model.
3. Repeat step 2 until the feature subset associated with the best model accuracy is found.

In an operative aspect of the present disclosure, by employing the BSWS on the extracted long-term acoustic features, the circuitry 108 is configured to create the set B 118 of the long-term acoustic features. Further, the BSWS is also configured to create the set C 120, which includes the features associated with the set B 118 of the long-term acoustic features in combination with features associated with the set A 116 of short-term acoustic features.

As shown by block 210 in FIG. 2, a RF model 114 is created by using the acoustic features of the set A 116, set B 118, and set C 120 as the training set. The RF model 114 is configured to classify healthy patients and neurodegenerative diseases patients based on the voice signals received from the patient/human. In an aspect, the RF model 114 is created by:
dividing the first plurality of voice signals into a training set and a test set of voice signals;
building the RF model 114 using bootstrap sampling of the training set wherein the model comprises multiple decision trees produced by multiple training subsets; and
testing the model with the test set of voice signals.

FIG. 3 illustrates a method 300 of extracting coefficients of the Mel spectrum, according to aspects of the present disclosure. To extract MFCCs, a number of steps may be followed as shown in FIG. 3.

### a) Framing (302)

The sampled voice signal is broken down into a plurality of overlapping frames, where each frame includes N samples. The voice signal is framed into short windows with an assumption that signal characteristics in the specified frame length are stationary, and therefore, mis-representations due to the rapidly varying nature of human voice signals are eliminated. The number of samples N is determined by N=Fₛ×frame length in seconds. There may be an overlapping between 30-50% of the frame samples and the frame length is set to 20-40 ms.

### b) Windowing (304)

Due to framing, signal discontinuities may result in high frequency noise at the edges of the frame, therefore, to reduce the edge effect and signal discontinuities, each frame is multiplied by a Hanning window of length equal to N. The mathematical representation of the Hanning window is expressed in equation 1:

$$w [ n ] = \frac{1}{2} [ 1 - cos ( \frac{2 π n}{N} ) ] ; 0 ⩽ n ⩽ N \qquad ( 1 )$$

where N is the number of filterbanks.

If the window is defined as w[n], and N is the number of samples per frame, then the windowed signal y[n] is given in equation 2:

y[n]=x[n]w[n];0≤n≤N. (2)

### c) Fast Fourier Transform (306)

Fast Fourier transform (FFT) is applied to convert the voice signal into frequency domain and to calculate periodogram of the voice signal as the square of the FFT spectrum. If the FFT is calculated using equation 3, then the periodogram is calculated as

$${S}_{n} = {\sum }_{k = 0}^{N - 1} {s}_{k} {e}^{\frac{- 2 π jkn}{N}} ; n = 0 , 1 , 2 , … , N - 1. \qquad ( 3 )$$

### d) Mel Filterbank (310)

MFCCs models the natural auditory functions of humans using logarithms and Mel scale. The human ear hears sounds approximately linearly up to 1 kHz, and logarithmically for higher frequencies. The Mel filterbanks (310) are a set of triangular bandpass filters overlapped by 50% and spaced linearly using Mel scale. The Mel filterbanks (310) are used to model the mechanism of human auditory function. Thus, the spectral power density contained in each filter bandwidth is averaged to obtain one value from each Mel filter.

The logarithms (308) of the average values are calculated to generate the cepstrum and consequently model the signal in cepstral domain. The spacing between the Mel filterbanks (310) is determined using the Mel scale. The conversion from frequency (Hz) to perceived frequency (Mel) is performed using equation 4:

$$Mel ( f ) = 2595 {log}_{10} ( 1 + \frac{f}{700} ) \qquad ( 4 )$$

The linearly spaced Mel filterbanks (310) is calculated using equation 4, then converted back to frequency domain using equation 5 given as below:

$$f ( Mel ) = 700 exp ( \frac{Mel}{1125} - 1 ) , \qquad ( 5 )$$

### e) Discrete Cosine Transform (DCT) (312)

DCT attempts to solve the correlation between energy log values obtained from the cepstrum. Then, these values are converted from cepstral to temporal domain in order to be classified using the RF model 114 before obtaining the MFCC. The DCT (312) is performed using equation 6 as follows:

$${c}_{i} = \sqrt{\frac{2}{N}} {\sum }_{j = 1}^{N} {m}_{j} cos ( \frac{π i}{N} ( j - 0.5 ) ) \qquad ( 6 )$$

where mⱼ is the log filterbank amplitudes and N is the number of Mel filterbanks.

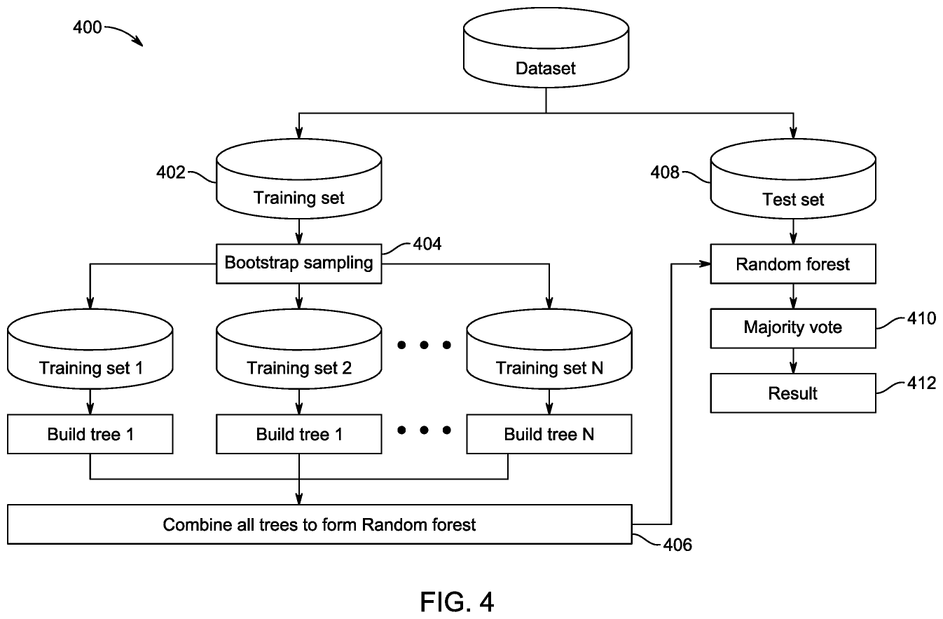

FIG. 4 represents a schematic working of the RF model 114, according to aspects of the present disclosure. The RF model 114 is one of robust classifiers used for PD detection along with support vector machine (SVM) and k nearest neighbor. Compared to other existing supervised learning classifiers, the RF model 114 exhibits more resistance to over—and underfitting and less sensitivity to outliers, with relatively fewer hyper-parameters. The RF model 114 requires splitting the dataset into a training set 402, and a test set 408, where the training set 402 is used to build the model and the test set 408 is used to test the model's performance. As the name implies, the RF model 114 applies bootstrap sampling 404 to produce multiple decision trees (DT) which are produced by n training subsets, as illustrated in FIG. 4.

The hyper-parameter n is indicative of the number of DT constituting the RF model 114. In an aspect, each of the training set is configured to generate a build tree. Further, all the generated build trees are combined to form a random forest, as shown by block 406 in FIG. 4. Typically, a larger random forest leads to a more robust performance. In each bootstrap set, some randomly chosen observations referred to as Out-Of-Bag (OOB) samples do not participate in tree training, instead, OOB are used as unseen test data to estimate the OOB error of each grown DT. The combination of parameters producing the smallest OOB error is chosen for classification. After building the RF model 114, the observations in the test set 408 which are unknown to the RF model 114 are evaluated and each DT in the forest produces a vote and the majority vote is selected as the forest final classification (as shown by block 410). In an aspect, the number of trees n in the RF model 114 are used for classifications was set to 100 trees. Further, to train and test the RF model 114, the data are split into the training data and the testing data. In an exemplary aspect, the division scheme has been applied where 75% of the dataset has been used for training the RF model 114 and the remaining 25% is used to test the performance of the trained RF model. Further, a 5-fold cross validation algorithm has been applied to obtain the prediction and test the accuracy.

In an operative aspect, the present system 100 is configured to obtain a second plurality of voice signals from humans of undetermined health status. The present system 100 is configured to apply the second plurality of voice signals against the RF model 114 in order to determine which patients in the second plurality of voice signals are healthy patients and which patients have neurodegenerative disease (as shown by block 412).

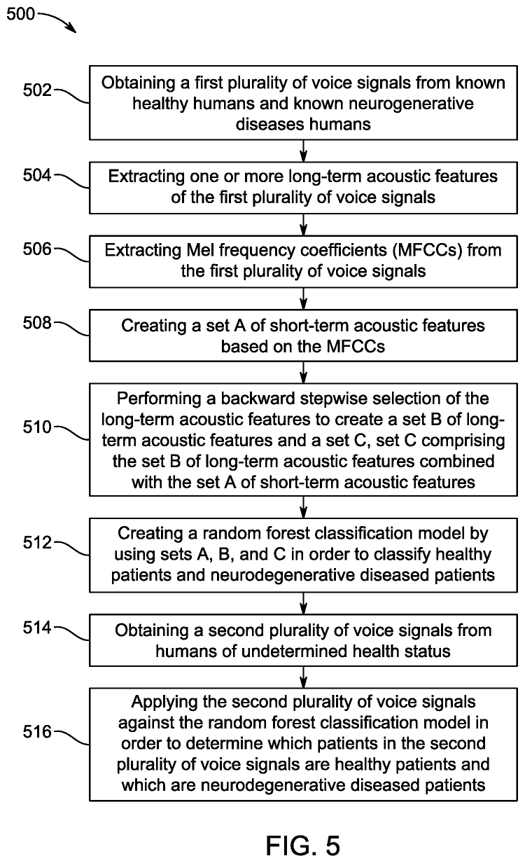

FIG. 5 illustrates a machine-learning method to differentiate between patients with neurodegenerative disease and healthy patients, according to one or more aspects.

Step 502 includes obtaining a first plurality of voice signals from known healthy humans and known neurogenerative diseases humans. In an aspect, the microphone 102 is configured to receive an audio input from a human and to generate a voice signal. In another aspect, the microphone 102 may be configured to transmit the generated voice signal to the circuitry 108.

Step 504 includes extracting one or more long-term acoustic features of the first plurality of voice signals. According to aspects of the present disclosure, the circuitry 108 is configured to extract the acoustic features of the received voice signals. Two types of acoustic features are extracted during the feature extraction, namely long-term features and short-term features.

Step 506 includes extracting Mel frequency coefficients (MFCCs) from the first plurality of voice signals.

Step 508 includes creating a set A 116 of short-term acoustic features based on the MFCCs. According to aspects of the present disclosure, the circuitry 108 extracts MFCCs from the received first plurality of voice signals and creates a set A 116 of short-term acoustic features based on the extracted MFCCs.

Step 510 includes performing a backward stepwise selection of the long-term acoustic features. The backward stepwise selection (feature selection algorithm) is applied to the extracted acoustic features for selecting the optimal feature set. The circuitry 108 is configured to perform the backward stepwise selection of the long-term acoustic features to create a set B 118 of long-term acoustic features. After that, the circuitry 108 may be configured to perform the backward stepwise selection to create a set C 120, which includes the set B 118 of long-term acoustic features combined with the set A 116 of short-term acoustic features.

Step 512 includes creating a RF model 114 by using sets A, B, and C. In communication with the training module 110, the circuitry 108 is configured to create the RF model 114 by combining all features associated with set A 116, set B 118, and set C 120 in order to classify healthy patients and neurodegenerative diseases patients.

Step 514 obtaining a second plurality of voice signals from humans of undetermined health status. In an aspect, the circuitry 108 is configured to obtain the second plurality of voice signals, recorded by the microphone 102.

Step 516 includes applying the second plurality of voice signals against the RF model 114 in order to determine which patients in the second plurality of voice signals are healthy patients and which are neurodegenerative diseases patients.

### Examples and Experiments

The following examples are provided to illustrate further and to facilitate the understanding of the present disclosure.

To measure the success of the RF model 114 and evaluate the discriminant potential of the RF model 114 to differentiate between PWP and healthy patients, four statistical measures are used; accuracy, specificity, sensitivity, and area under the receiver operating characteristic (ROC) curve, namely AUC.

In an aspect, the circuitry 108 is additionally configured to determine an accuracy, a specificity, and a sensitivity of the RF model 114. The accuracy refers to the percentage of correctly classified samples. The accuracy may be calculated by:

$$\frac{TP + TN}{TP + TN + FP + FN} . \qquad ( 7 )$$

The specificity indicates the number of healthy subjects who were correctly classified. The specificity is calculated by:

$$\frac{TN}{TN + FP} . \qquad ( 8 )$$

The sensitivity is the percentage of PD patients who were correctly classified. The sensitivity is calculated by:

$$\frac{TP}{TP + FN} . \qquad ( 9 )$$

where:
True Positive (TP) indicates a number of correctly classified diseased patients;
True Negative (TN) expresses a number of correctly classified healthy patients;
False Positive (FP) indicates a number of incorrectly classified healthy subjects; and
False Negative (FN) expresses a number of incorrectly classified diseased patients.

Further, the ROC curve evaluates performance of the RF model 114 at various threshold values by plotting true positive rate (TPR) to false positive rate (FPR). In an aspect, the TPR is another term used to refer to the sensitivity, while term FPR is mathematically represented in

Equation 10 as follows:

$$FPR = \frac{FP}{TN + FP} . \qquad ( 10 )$$

Experimental Data and Analysis

In an aspect of the present disclosure, the training set is collected from an online open-source dataset that found in a ML repository of the University of California Irvine (UCI).

In an example, the sample recording took place at the department of neurology, Istanbul university with the approval of clinical research ethics committee of Bahcesehir. Two groups of people consented to participate in the dataset: a PD patients' group that consist of 188 individuals (107 males and 81 females) with ages ranging from 33 to 87 years old, and a control group that consists of 64 healthy individuals (23 males and 41 females) with ages ranging from 41 to 82 years old. Participated people were instructed to sustain the phonation of the vowel/a/10 centimeters away from the microphone 102 and three phonations from each subject were recorded collectively obtaining a total of 756 phonations.

The first step of the conducted experiments is to feed the extracted acoustic features into the developed feature selection module to reduce the dimensionality of feature subsets and subsequently reduce the computational resources required for selecting the optimal feature set. The first step utilizes the BSWS to obtain three sets; Set A 116, Set B 118, and Set C 120 and their feature count as shown in Table 1.

A tabular representation of a feature set obtained from BSWS is illustrated in Table 1 provided below.

TABLE 1
Feature sets obtained from backward stepwise selection

| | Feature | |
|---|---|---|
| Feature set | count | Description |
| Set A | 13 | 1^{st} MFCC-13^{th} MFCC |
| Set B | 10 | DFA, locAbsJitter, rapJitter, apq3Shimmer, |
| | | minIntensity, maxIntensity, Mean intensity, F1, F2, |
| | | Localpctjitter |
| Set C | 20 | DFA, locAbsJitter, rapJitter, apq3Shimmer, |
| | | minIntensity, maxIntensity, Mean intensity, F1, F2, |
| | | Localpctjitter 4^{th}, 6^{th}, and 11^{th} MFCC |

Set A 116 includes the short-term features, namely the first thirteen coefficients of the Mel cepstrum, therefore, BSWS was not used at this stage. Set B 118 includes long-term features obtained by feeding all extracted long-term features through the BSWS, while set C 120 is obtained by feeding a combination of sets A and B to the BSWS. The number of features of set B 118 is determined by exhaustive trials to reach the highest accuracy, which combined with the features of set A 116 yields 23 features. BSWS was performed to reduce the dimensionality of the feature vector and avoid the use of redundant features.

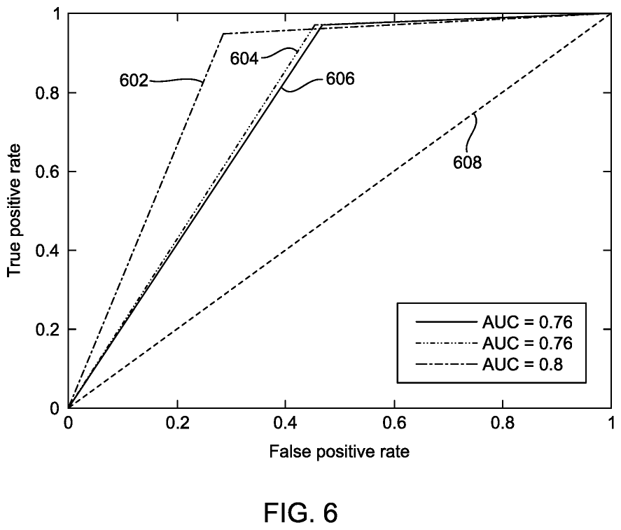

Table 2 shows the individual classification performances of the three feature sets using the RF model 114 and a 5-fold cross validation scheme. Set A 116 and Set B 118 exhibit relatively similar performances in terms of accuracy, specificity, and sensitivity. Such similarity illustrates the complementary inherent properties of MFCCs and long-term features. Short-term features of set A (MFCCs) 116 are less robust in noisy environments, but the inter-correlation of features is considerably low. On the other hand, long-term features of set B 118 are quite the opposite i.e., highly correlated with high tolerance to noisy signal counterparts. By using the BSWS, the intercorrelation perceived in long-term features is eliminated, and the recordings obtained from the dataset being marginally noise free, thus, the downfalls of each type of feature are alleviated. The combination of the short-term, and long-term features has proven to be highly effective, set C 120 is definitely less correlated and more robust in the presence of noise than sets A and B. Hence, set C 120 achieved the highest accuracy of 88.84%. While sensitivity is the percentage of correctly diagnosed PD patients, specificity measures the number of correctly diagnosed healthy subjects. Sensitivity and specificity are some of the metrics used to evaluate diagnostic tests, however, in some embodiments, in PD detection sensitivity is given more weightage than specificity. Unlike false positives, false negatives are susceptible to more neuronal damage, therefore, the performance of the RF model 114 is considered well, although specificity values are low. Specificity values obtained by the three sets are relatively low compared to sensitivity where the highest specificity value is obtained from set C 120. Sets A and B produced specificities that allowed only half of the healthy subjects to be correctly diagnosed. The dataset used to train and test the RF model 114 contained a total of 756 subjects of which 74.6% were PD patients. The low specificity values obtained are attributed to the vast gap in count between the control group and PD group which caused the overfitting of random forest; hence, most PD patients were correctly classified, and more healthy subjects were classified as PD patients. Receiver operating characteristics (ROC) curves for sets A, B, and C are represented in FIG. 6. The area under the curve for set A and set B is 0.76 (shown by reference numerals 604, and 606), and 0.8 for set C (shown by reference numeral 602). The AUC obtained with set C 120 is the largest and highlights the effect of the addition of short-term features to long-term features on the performance and discriminant potential of the RF model 114. FIG. 6 also indicates the degree of separability and the high prediction accuracy achieved by set C 120 when compared to sets A and B.

TABLE 2
Individual classification performances of the three feature sets

| | Total | Number of | Number of | | |
|---|---|---|---|---|---|
| | Number of | Training | Test | | |
| | Images | Images | Images | Accurate | Misclassified |
| BI 0 | 530 | 424 | 106 | 104 (98.1%) | 2 (1.9%) |
| BI 1-6 | 773 | 617 | 156 | 152 (97.4%) | 4 (2.6%) |
| Total | 1303 | 1041 | 262 | 256 (97.7%) | 6 (2.3%) |

The performance and effectiveness of the developed method are examined using a dataset of 756 voice samples. The results indicate that the combination of long-term features along with MFCCs in the input dataset considerably improves the PD detection system and increases the detection accuracy to 88.84%. They also illustrate the ability of the developed method to predict PD patients with a sensitivity of 98.51%. In addition to that, the results show considerable improvement of approximately 30% in the specificity value with 71.08% for the combined set (C) as compared to MFCCs set (A) and long-term features set (B) with specificity values of 53.7% and 55% respectively.

Thus, the implementation of the developed method considerably improves the PD detectability at early stages, which allows for proactive and preventative medical treatment that may help in alleviating and potentially preventing the disease consequences at a later stage

An embodiment is illustrated with respect to FIGS. 1-6. The another embodiment describes a non-transitory computer-readable storage medium storing computer-readable instructions that, when executed by one or more processors 106, cause the one or more processors 106 to obtain a first plurality of voice signals from human patients, extract one or more long-term acoustic features of the voice signals, extract Mel frequency coefficients (MFCCs) from the voice signals, creating a set A 116 of short-term acoustic features based on the MFCCs, perform a backward stepwise selection of long-term acoustic features to create a set B 118 of long term acoustic features and a set C 120, set C 120 comprising long-term acoustic features of set B combined with the set A 116 of short-term acoustic features, create a RF model 114 by using sets A, B, and C in order to create a classification of healthy patients and neurodegenerative diseases patients, obtaining a second plurality of voice signals, and apply the second plurality of voice signals against the model in order to determine which of the second plurality of voice signals are from healthy patients and which are from neurodegenerative diseases patients.

In an aspect, the computer-readable instructions further calculate accuracy, specificity, and sensitivity of the RF model 114 as previously described by equations (7), (8), and (9).

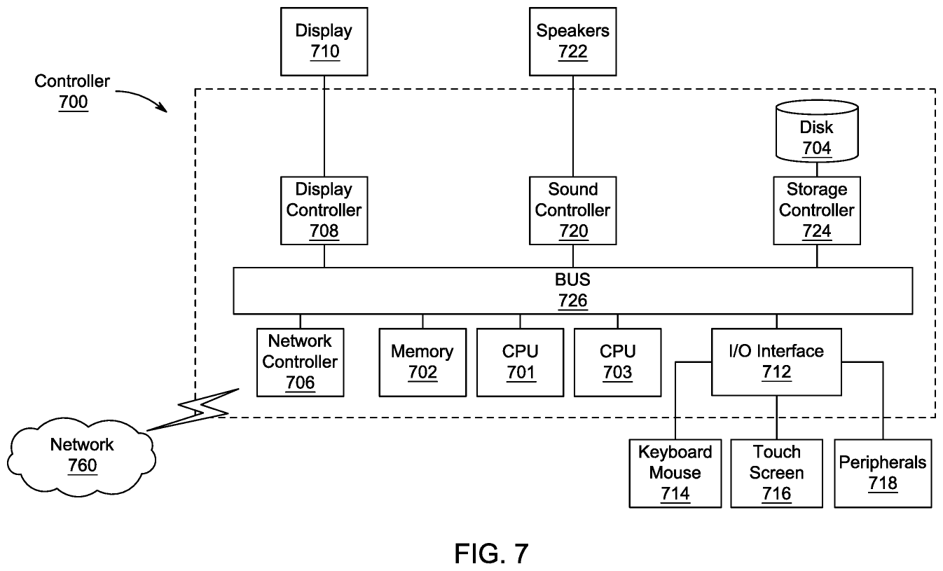

Next, further details of the hardware description of the computing environment of FIG. 1 according to exemplary embodiments are described with reference to FIG. 7. In FIG. 7, a controller 700 is described is representative of the processor (s) 106 and the circuitry 108 of FIG. 1 in which the circuitry 108 is a computing device that includes a CPU 701 which performs the processes described above/below. The process data and instructions may be stored in memory 702. These processes and instructions may also be stored on a storage medium disk 704 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 701, 703 and an operating system such as Microsoft Windows 9, Microsoft Windows 10, UNIX, Solaris, LINUX, Apple MAC-OS and other systems known to those skilled in the art.

The hardware elements in order to achieve the computing device may be realized by various circuitry elements, known to those skilled in the art. For example, CPU 701 or CPU 703 may be a Xenon or Core processor from Intel of America or an Opteron processor from AMD of America or may be other processor types that would be recognized by one of ordinary skill in the art. Alternatively, the CPU 701, 703 may be implemented on an FPGA, ASIC, PLD or using discrete logic circuits, as one of ordinary skill in the art would recognize. Further, CPU 701, 703 may be implemented as multiple processors cooperatively working in parallel to perform the instructions of the inventive processes described above.

The computing device in FIG. 7 also includes a network controller 706, such as an Intel Ethernet PRO network interface card from Intel Corporation of America, for interfacing with network 760. As can be appreciated, the network 760 can be a public network, such as the Internet, or a private network such as an LAN or WAN network, or any combination thereof and can also include PSTN or ISDN sub-networks. The network 760 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The computing device further includes a display controller 708, such as a NVIDIA GeForce GTX or Quadro graphics adaptor from NVIDIA Corporation of America for interfacing with display 710, such as a Hewlett Packard HPL2445w LCD monitor. A general purpose I/O interface 712 interfaces with a keyboard and/or mouse 714 as well as a touch screen panel 716 on or separate from display 1110. General purpose I/O interface also connects to a variety of peripherals 718 including printers and scanners, such as an OfficeJet or DeskJet from Hewlett Packard.

A sound controller 720 is also provided in the computing device such as Sound Blaster X-Fi Titanium from Creative, to interface with speakers/microphone 722 thereby providing sounds and/or music.

The general purpose storage controller 724 connects the storage medium disk 704 with communication bus 726, which may be an ISA, EISA, VESA, PCI, or similar, for interconnecting all of the components of the computing device. A description of the general features and functionality of the display 710, keyboard and/or mouse 714, as well as the display controller 1108, storage controller 724, network controller 706, sound controller 720, and general purpose I/O interface 712 is omitted herein for brevity as these features are known.

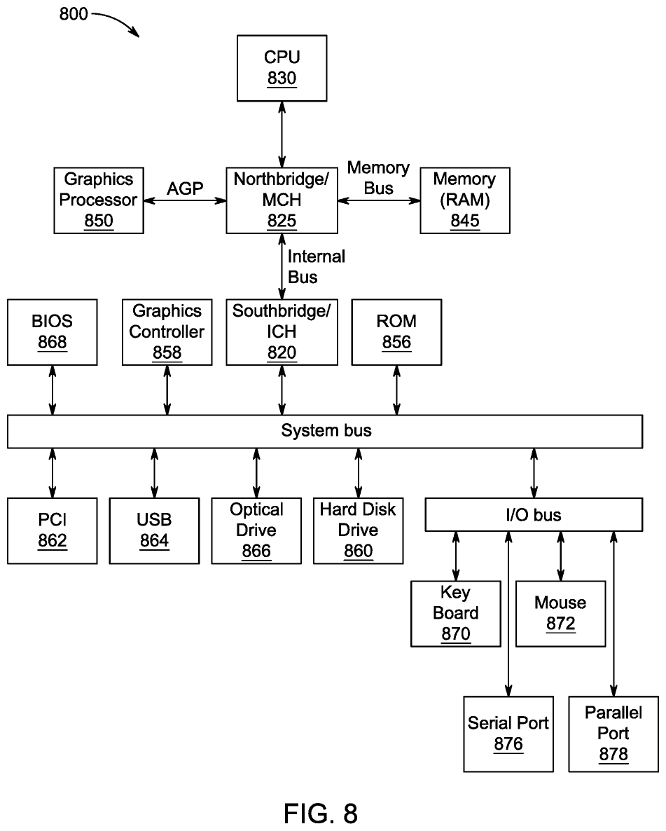

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 8.

FIG. 8 shows a schematic diagram of a data processing system 800 used within the computing system, according to exemplary aspects of the present disclosure. The data processing system 800 is an example of a computer in which code or instructions implementing the processes of the illustrative aspects of the present disclosure may be located.

In FIG. 8, data processing system 800 employs a hub architecture including a north bridge and memory controller hub (NB/MCH) 825 and a south bridge and input/output (I/O) controller hub (SB/ICH) 820. The central processing unit (CPU) 830 is connected to NB/MCH 825. The NB/MCH 825 also connects to the memory 845 via a memory bus and connects to the graphics processor 850 via an accelerated graphics port (AGP). The NB/MCH 825 also connects to the SB/ICH 820 via an internal bus (e.g., a unified media interface or a direct media interface). The CPU Processing unit 830 may contain one or more processors and even may be implemented using one or more heterogeneous processor systems.

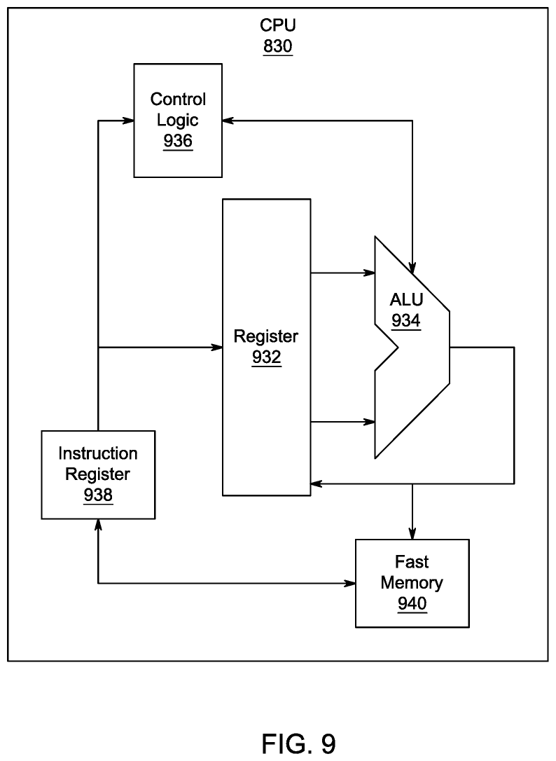

For example, FIG. 9 shows one aspects of the present disclosure of CPU 830. In one aspects of the present disclosure, the instruction register 938 retrieves instructions from the fast memory 940. At least part of these instructions is fetched from the instruction register 938 by the control logic 936 and interpreted according to the instruction set architecture of the CPU 830. Part of the instructions can also be directed to the register 932. In one aspects of the present disclosure the instructions are decoded according to a hardwired method, and in other aspects of the present disclosure the instructions are decoded according to a microprogram that translates instructions into sets of CPU configuration signals that are applied sequentially over multiple clock pulses. After fetching and decoding the instructions, the instructions are executed using the arithmetic logic unit (ALU) 934 that loads values from the register 932 and performs logical and mathematical operations on the loaded values according to the instructions. The results from these operations can be feedback into the register and/or stored in the fast memory 940. According to certain aspects of the present disclosures, the instruction set architecture of the CPU 830 can use a reduced instruction set architecture, a complex instruction set architecture, a vector processor architecture, a very large instruction word architecture. Furthermore, the CPU 830 can be based on the Von Neuman model or the Harvard model. The CPU 830 can be a digital signal processor, an FPGA, an ASIC, a PLA, a PLD, or a CPLD. Further, the CPU 830 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 8, the data processing system 800 can include that the SB/ICH 820 is coupled through a system bus to an I/O Bus, a read only memory (ROM) 856, universal serial bus (USB) port 864, a flash binary input/output system (BIOS) 868, and a graphics controller 858. PCI/PCIe devices can also be coupled to SB/ICH 820 through a PCI bus 862.

The PCI devices may include, for example, Ethernet adapters, add-in cards, and PC cards for notebook computers. The Hard disk drive 860 and CD-ROM 856 can use, for example, an integrated drive electronics (IDE) or serial advanced technology attachment (SATA) interface. In one aspects of the present disclosure, the I/O bus can include a super I/O (SIO) device.

Further, the hard disk drive (HDD) 860 and optical drive 866 can also be coupled to the SB/ICH 820 through a system bus. In one aspects of the present disclosure, a keyboard 870, a mouse 872, a parallel port 878, and a serial port 876 can be connected to the system bus through the I/O bus. Other peripherals and devices that can be connected to the SB/ICH 820 using a mass storage controller such as SATA or PATA, an Ethernet port, an ISA bus, an LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

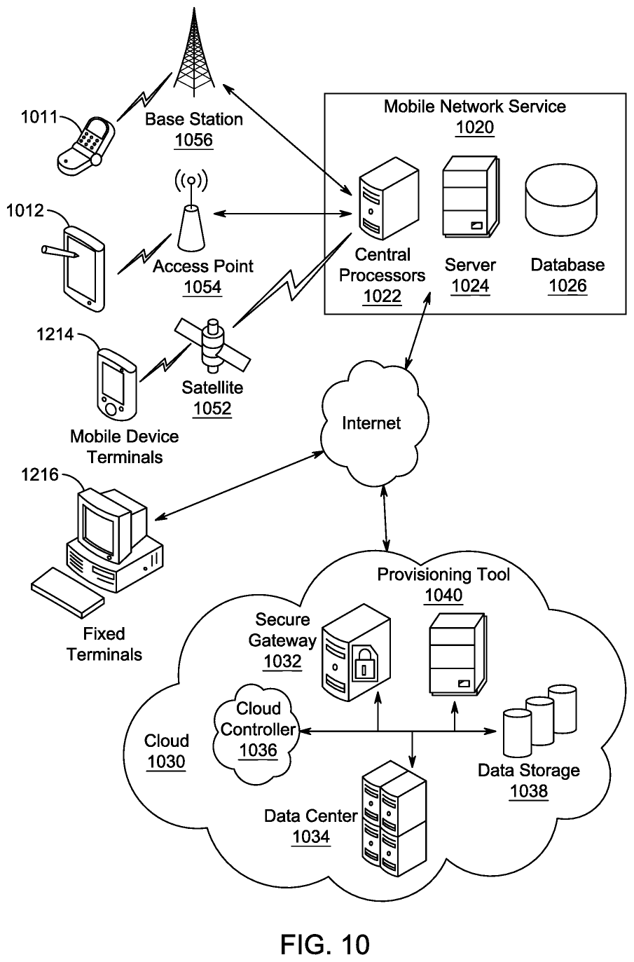

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 10, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely, either in real-time or as a batch process. Additionally, some aspects of the present disclosures may be performed on modules or hardware not identical to those described. Accordingly, other aspects of the present disclosures are within the scope that may be claimed. More specifically, FIG. 10 illustrates client devices including smart phone 1011, tablet 1012, mobile device terminal 1014 and fixed terminals 1016. These client devices may be coupled with a mobile network service 1020 via base station 1056, access point 1054, satellite 1052 or via an internet connection. Mobile network service 1020 may comprise central processors 1022, server 1024 and database 1026. Fixed terminals 1016 and mobile network service 1020 may be coupled via an internet connection to functions in cloud 1030 that may comprise security gateway 1032, data center 1034, cloud controller 1036, data storage 1038 and provisioning tool 1040.

The above-described hardware description is a non-limiting example of corresponding structure for performing the functionality described herein.

Obviously, numerous modifications and variations of the present disclosure are possible in light of the above teachings. It is therefore to be understood that within the scope of the appended claims, the invention may be practiced otherwise than as specifically described herein.

1. A machine-learning method to differentiate between patients with neurodegenerative disease and healthy patients, the method comprising:
obtaining a first plurality of voice signals from known healthy humans and known neurogenerative diseased humans;
extracting one or more long-term acoustic features of the first plurality of voice signals;
extracting Mel frequency coefficients (MFCCs) from each of the first plurality of voice signals;
creating a set A of short-term acoustic features based on the MFCCs;
performing a backward stepwise selection of the long-term acoustic features to obtain a set B of long-term acoustic features and a set C, set C comprising the set B of long-term acoustic features combined with the set A of short-term acoustic features;
configuring a random forest classification model with the features of set C in order to classify healthy patients and neurodegenerative disease patients;
obtaining a second plurality of voice signals from humans of undetermined health status; and
applying the second plurality of voice signals against the random forest classification model in order to determine which patients in the second plurality of voice signals are healthy patients and which are neurodegenerative disease patients.

2. The method according to claim 1, which further comprises determining an accuracy, a specificity, and a sensitivity of the random forest classification model wherein the accuracy is calculated by:
$$\frac{TP + TN}{TP + TN + FP + FN}$$
the specificity is calculated by:
$$\frac{TN}{TN + FP}$$
and the sensitivity is calculated by
$$\frac{TP}{TP + FN}$$
where TP is true positive (TP) indicates a number of correctly classified diseased patients and true negative (TN) expresses a number of correctly classified healthy patients and false positive (FP) indicates a number of incorrectly classified healthy subjects, and false negative (FN) expresses a number of incorrectly classified diseased patients.

3. The method according to claim 1, wherein the random forest classification model is created by:
dividing the first plurality of voice signals into a training set and a test set of voice signals;
building the random forest classification model using bootstrap sampling of the training set wherein the model comprises multiple decision trees produced by multiple training subsets; and
testing the model with the test set of voice signals.

4. The method of claim 1, wherein the Mel frequency coefficients are extracted by a method comprising:
dividing the voice signals into overlapping frames, each frame containing a plurality of samples wherein the overlap is between 30% and 50% of the frame;
windowing the overlapping frames wherein the window is of length 20-40 ms;
applying a Fast Fourier Transform (FFT) to convert the voice signal to a frequency domain;
calculating logarithm of an average value of a spectral power density in each of the frames to model the voice signal in a cepstral domain;
creating Mel filterbanks within the cepstral domain; and
performing a discrete cosine transformation (DCT) on the Mel filterbanks to determine the Mel frequency coefficients.

5. The method of claim 1, wherein the backward stepwise selection of the long-term acoustic features comprises:
starting with a model with a full set of long-term acoustic features;
iteratively removing a particular feature that has the least significance for model accuracy;
removing the particular feature from the model when removal of the particular feature from the model improves model performance wherein performance is measured by accuracy, specificity, sensitivity, or an area under a curve;
returning the particular feature to the model when removing the particular feature worsens the model performance; and
repeating removal of each feature in the set of long-term acoustic features until the best performance of the model is achieved as measured by accuracy, a specificity, a sensitivity, or an area under the curve.

6. The method of claim 1, wherein the long-term acoustic features comprise any of:
a relative average perturbation;
a jitter;
an amplitude perturbation quotient;
a shimmer;
a detrended fluctuation analysis;
a minimum intensity;
a maximum intensity;
a mean intensity; and
a formant frequency.

7. The method of claim 1 wherein the neurodegenerative disease is Parkinson's disease.

8. A medical diagnostic system, comprising:
one or more processors,
a memory,
a microphone, and
a circuitry configured to:
obtain a first plurality of voice signals from known healthy humans and known neurogenerative diseases humans;
extract one or more long-term acoustic features of the first plurality of voice signals;
extract Mel frequency coefficients (MFCCs) from the first plurality of voice signals;
create a set A of short-term acoustic features based on the MFCCs;
perform a backward stepwise selection of the long-term acoustic features to create a set B of long-term acoustic features and a set C, set C comprising the set B of long-term acoustic features combined with the set A of short-term acoustic features;
configuring a random forest classification model with set the features of set C in order to classify healthy patients and neurodegenerative diseased patients;
obtain a second plurality of voice signals from humans of undetermined health status; and
apply the second plurality of voice signals against the model in order to determine which patients in the second plurality of voice signals samples are healthy patients and which are neurodegenerative diseased patients.

9. The medical diagnostic system of claim 8, wherein the circuitry is additionally configured to determine an accuracy, a specificity, and a sensitivity of the random forest classification model wherein the accuracy is calculated by:
$$\frac{TP + TN}{TP + TN + FP + FN}$$
the specificity is calculated by:
$$\frac{TN}{TN + FP}$$
and the sensitivity is calculated by
$$\frac{TP}{TP + FN}$$
where TP is true positive (TP) indicates a number of correctly classified diseased patients and true negative (TN) expresses a number of correctly classified healthy patients and false positive (FP) indicates a number of incorrectly classified healthy subjects, and false negative (FN) expresses a number of incorrectly classified diseased patients.

10. The medical diagnostic system of claim 8, wherein the random forest classification model is created by:
dividing the first plurality of voice signals into a training set and a test set of voice signals;
using bootstrap sampling of the training set wherein the model comprises multiple decision trees produced by multiple training subsets; and
testing the random forest classification model with the test set of voice signals.

11. The medical diagnostic system of claim 8, wherein the circuitry is configured to extract the MFCCs by:
dividing the voice signal into overlapping frames, each frame containing a plurality of samples wherein the overlap is between 30% and 50% of the frame;
windowing the overlapping frames where the window is 20-40 ms;
applying a Fast Fourier Transform (FFT) to convert the voice signal to a frequency domain;
calculating logarithms of average values of a spectral power density in each of the frames to model the voice signal in a cepstral domain;
creating Mel filterbanks within the cepstral domain; and
performing a discrete cosine transformation (DCT) on the Mel filterbanks to calculate the MFCCs.

12. The medical diagnostic system of claim 8, wherein the circuitry is configured to calculate the backward stepwise selection of the long-term acoustic features by:
starting with a model with a full set of long-term acoustic features;
iteratively removing a particular feature that has the least significance for model accuracy;
determining if a removal of a particular feature resulted in improving model performance wherein performance is measured by an accuracy, a specificity, a sensitivity, or an area under a curve, and removing the particular feature from the model;
determining if a removal of the particular feature resulted in worsening the model's performance and returning the particular feature to the model; and
repeating a removal of each feature in the set until the best model accuracy is found.

13. The medical diagnostic system of claim 8, wherein the long-term acoustic features comprise any of:
a relative average perturbation;
a jitter;
an amplitude perturbation quotient;
a shimmer;
a detrended fluctuation analysis;
a minimum intensity;
a maximum intensity;
a mean intensity; and
a formant frequency.

14. The medical diagnostic system of claim 8, wherein the neurodegenerative disease is Parkinson's disease.

15. A non-transitory computer-readable storage medium storing computer-readable instructions that, when executed by one or more processors, cause the one or more processors to:
obtain a first plurality of voice signals from human patients;
extract one or more long-term acoustic features of the voice signals;
extract Mel frequency coefficients (MFCCs) from the voice signals;
create a set A of short-term acoustic features based on the MFCCs;
perform a backward stepwise selection of long-term acoustic features to create a set B of long term acoustic features and a set C, set C comprising long-term acoustic features combined with the set A of short-term acoustic features;
configure a random forest classification model with the features of set C in order to create a classification of healthy patients and neurodegenerative diseased patients;
obtain a second plurality of voice signals; and
apply the second plurality of voice signals against the model in order to determine which of the second plurality of voice signals are from healthy patients and which are from neurodegenerative diseased patients.

16. The non-transitory computer-readable storage medium according to claim 15, wherein the computer-readable instructions further calculate an accuracy, a specificity, and a sensitivity of the random forest classification model wherein the accuracy is calculated by:
$$\frac{TP + TN}{TP + TN + FP + FN}$$
the specificity is calculated by:
$$\frac{TN}{TN + FP}$$
and the sensitivity is calculated by
$$\frac{TP}{TP + FN}$$
where TP is true positive (TP) indicates a number of correctly classified diseased patients and true negative (TN) expresses a number of correctly classified healthy patients and false positive (FP) indicates a number of incorrectly classified healthy subjects, and false negative (FN) expresses a number of incorrectly classified diseased patients.

17. The non-transitory computer-readable storage medium according to claim 15, wherein the computer-readable instructions to create a random forest classification model comprise:
dividing the first plurality of voice signals into a training set and a test set of voice signals;
using bootstrap sampling of the training set wherein the model comprises multiple decision trees produced by multiple training subsets; and
testing the model with the test set of voice signals.

18. The non-transitory computer-readable storage medium according to claim 15, wherein the computer-readable instructions to extract a Mel frequency coefficient comprise:
framing the voice signal into overlapping frames, each frame containing a plurality of samples wherein the overlap is between 30% and 50% of the frame;
windowing the overlapping frames where the window is 20-40 ms;
applying a Fast Fourier Transform (FFT) to convert the voice signal to a frequency domain;
calculating logarithms of average values of a spectral power density in each of the frames to model the voice signal in a cepstral domain;
creating Mel filterbanks within the cepstral domain; and
performing a discrete cosine transformation (DCT) on the Mel filterbanks to calculate the MFCC.

19. The non-transitory computer-readable storage medium according to claim 15, wherein long-term acoustic features comprise any of:
a relative average perturbation;
a jitter;
an amplitude perturbation quotient;
a shimmer;
a detrended fluctuation analysis;
a minimum intensity;
a maximum intensity;
a mean intensity; and
a formant frequency.

20. The non-transitory computer-readable storage medium according to claim 15, wherein the neurodegenerative disease is Parkinson's disease.
